(19) United States
(12) Patent Application Publication
(10) Pub. No.: US 2003/0157142 A1
(43) Pub. Date: Aug. 21, 2003

(54) Implants with a phosphazene-containing coating

(76) Inventors: Stefan Nagel, Berlin (DE); Michael Boxberger, Berlin (DE)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/344,216

(22) PCT Filed: Aug. 1, 2001
(86) PCT No.: PCT/EP01/08913

(30) Foreign Application Priority Data
Aug. 11, 2000 (EP) 00117191.7

(51) Int. Cl. A61K 48/00; A61K 31/366

(52) U.S. Cl. 424/423; 514/44; 514/460

(57) ABSTRACT

The invention relates to artificial implants with a biocompatible surface which has antithrombitic properties, further-more contains a pharmacologically active ingredient and a method for production thereof.

The present invention relates to artificial implants with a biocompatible coating having antithrombogenic properties and which also contains a pharmacologically active agent, as well as a process for their production.

The most serious complications caused by artificial implants are considered to be the increased deposition of thrombocytes on the exogenous surface. Such thrombi formation on contact of human blood with the exogenous surface, such as artificial heart valves, is described at the state of the art (cf. information material from the company Metronic Hall, Bad Homburg, Carmeda BioActive Oberfläche [Carmeda BioActive Surface] (CBSA), pages 1-21; B. D. Ratner, “The Blood Compatibility Catastrophe”, J. of Biomed. Mat. Res., Vol. 27, 283-287; and C. W. Akins, “Mechanical Cardiac Valvular Prostheses”, The Society of Thoracic Surgeons, 161-171 (1991)). For example, artificial heart valves found on the world market are made of pyrolyzed carbon and exhibit an increased tendency for development of thrombi (cf. C. W. Akins, above).

The polymeric compound poly[bis(trifluoroethoxy)phosphazene] was used to coat artificial implants in DE-C-19613048. Its effective antithrombogenic action was known from Holleman Wiberg, “Stickstoffverbindungen des Phosphors” [Nitrogen Compounds of Phosphorus], Lehrbuch der anorganischen Chemie [Textbook of Inorganic Chemistry], 666-669, 91^{st}-100^{th} Edition, Walter de Gruyter Verlag (1985), and from Tur, Vinogradova, et al., “Entwicklungstendenzen bei Polymeranalogen Umsetzungen von Polyphosphazen” [Tendencies in development of polymer-like reactions of polyphosphazenes], Acta Polymerica 39, 424-429, No. 8, (1988). Specifically, DE-C-19613048 describes an artificial implant comprising an implant material as the substrate and a biocompatible coating applied at least partly to the surface of the substrate, which coating contains an antithrombogenic polymer having the following general formula (I):

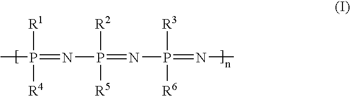

wherein R¹ to R⁶ are the same or different and represent an alkoxy, alkylsulfonyl, dialkylamino or aryloxy group, or a heterocycloalkyl or heteroaryl group having nitrogen as the heteroatom; it also describes methods of producing such artificial implants.

A problem with implants such as heart valves and stents (see DE-A-197 53 123), independently of whether the implant is coated with the present antithrombogenic material, is their tendency to restenosis, i.e., narrowing due to proliferation of smooth muscle cells in the vessel wall as a biological response to the implant. A survey article by Swanson and Gershlick (Stent, Vol. 2, 66-73 (1999)) mentions numerous approaches to the application of suitable active agents to the implants. These include the use of polymer-coated stents, suggested on page 68, wherein the polymer can act as a reservoir for active agents. However, it is immediately advised that this approach not be pursued, because an elevated tendency to inflammation was found in vivo in a test study in which stents were coated with various biodegradable polymers, all of them otherwise known to be biocompatible in vitro. Furthermore, U.S. Pat. Nos. 5,788,979 and 5,980,972 describe coating of materials with biodegradable polymers, in which the coating can also contain pharmacologically active agents.

An alternative approach to preventing excessive cell proliferation and the formation of scares is described in WO 99/16477. In this case, a radioactively labeled polymer of formula (I), above, preferably a polymer containing a radioactive isotope of phosphorus, is applied to the implant. The radioactive radiation emitted (β-radiation with ³²P) is said to prevent uncontrolled cell growth, which results in restenosis on stent implantation, for instance. Of course, when radioactive materials are used, safety requirements and side effects must be considered that stand in the way of the straightforward use of such implants.

Therefore, the object of the present invention is to provide artificial implants having not only outstanding mechanical properties but also antithrombogenic and anti-restenosis properties so as to improve the biocompatibility and tolerability of such implants. Further, it is another object of the present invention to provide processes for the production of such implants.

It was found, surprisingly, that the polymer of formula (I) defined above exhibits outstanding matrix properties for pharmacologically active agents, and when these active agents are applied to an implant material, the polymer delivers them to its surroundings in a controlled manner. It was also found, surprisingly, that there is no inflammatory reaction on biological degradation of the polymer of formula (I). This makes possible a controlled release of active agent, not only through diffusion and dissolution processes, but also through biological degradation of the matrix and the associated release of incorporated active agents without occurrence of an undesired inflammatory reaction.

The present invention relates to an artificial implant comprising an implant material as the substrate and a biocompatible coating applied at least partly to the substrate surface, which coating comprises an antithrombogenic polymer having the following general formula (I)

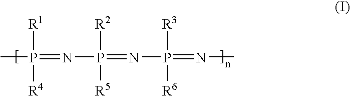

wherein R¹ to R⁶ are the same or different and represent an alkoxy, alkylsulfonyl, dialkylamino or aryloxy group, or a heterocycloalkyl or heteroaryl group having nitrogen as the heteroatom, and at least one other (additional) pharmacologically active agent (briefly, “active agent” in the following).

In the polymer of formula (I) it is preferable for at least one of the groups R¹ to R⁶ to be an alkoxy group substituted with at least one fluorine atom.

In the polymer of formula (I), the alkyl groups in the alkoxy, alkylsulfonyl and dialkylamino groups are, for example, straight-chain or branched-chain alkyl groups having 1 to 20 carbon atoms, wherein the alkyl groups can be substituted, for example, with at least one halogen atom, such as a fluorine atom.

Examples of alkoxy groups are methoxy, ethoxy, propoxy and butoxy groups, which preferably can be substituted with at least one fluorine atom. The 2,2,2-trifluoroethoxy group is particularly preferred.

Examples of alkylsulfonyl groups are methylsulfonyl, ethylsulfonyl, propylsulfonyl and butylsulfonyl groups.

Examples of dialkylamino groups are dimethylamino, diethylamino, dipropylamino and dibutylamino groups.

The aryl group in the aryloxy group is, for instance, a compound having one or more aromatic ring systems, wherein the aryl group can be substituted, for instance, with at least one alkyl group as defined above.

Examples of aryloxy groups are phenoxy and naphthoxy groups and derivatives of them.

The heterocycloalkyl group is, for example, a ring system containing 3 to 7 atoms, at least one of the ring atoms being a nitrogen atom. The heterocycloalkyl group can, for example, be substituted with at least one alkyl group as defined above. Examples of heterocycloalkyl groups are piperidinyl, piperazinyl, pyrrolidinyl and morpholinyl groups and their derivatives.

The heteroaryl group is, for example, a compound with one or more aromatic ring systems, wherein at least one ring atom is a nitrogen atom. The heteroaryl group can, for example, be substituted with at least one alkyl group as defined above. Examples of heteroaryl groups are pyrrolyl, pyridinyl, pyridinolyl, isoquinolinyl and quinolinyl groups and their derivatives.

In a preferred embodiment of the present invention, the biocompatible coating contains the antithrombogenic polymer poly[bis(trifluoroethoxy)phosphazene].

The other pharmacologically active agent is preferably an organic (low or higher molecular weight) compound, especially an antimitogenic active agent such as a cytostatic (such as paclitaxel etc.), a PDGF inhibitor (such as tyrphostins etc.), a Raf-1 kinase inhibitor, a monoclonal antibody for integrin blockade of leukocytes, an antisense active agent (such as plasmid DNA etc.), superoxide dismutase, a radical trap (such as probucol etc.), a steroid, a statin (such as cerivastatin etc.), a corticosteroid (such as methotrexate, dexamethasone, methylprednisolan [sic] etc.), an adenylate cyclase inhibitor (such as forskolin etc.), a somatostatin analogue (such as angiopeptin etc.), an antithrombin agent (such as argatroban etc.), a nitric oxide donor, a glycoprotein IIb/IIIa receptor antagonist (such as urokinase derivatives, abciximab, tirofiban etc.), an antithrombotic agent (such as activated protein C, PEG-hirudin, prostaglandin analogues etc.), a vascular endothelial growth factor (VEGF), trapidil etc., and mixtures of these.

It is desirable that the content of active agent in the biocompatible coating be as high as possible to prevent restenosis effectively. It has been shown that the coating may contain up to 50% by weight of active agent without significant damage to the mechanical properties of said coating. According to the invention, the proportion of active agent in the coating is in the range of 0.01 to 50% by weight, and preferably 0.2 to 30% by weight. This is approximately equivalent to a polymer to active agent weight ratio of 1:0.0001 to 1:1, preferably 1:0.05 to 1:0.5.

The biocompatible coating of the artificial implant according to the invention has, for example, a thickness of 1 nm to about 100 μm, preferably 10 nm to 10 μm, and especially preferred up to about 1 μm.

There is no particular limit to the implant material used as the substrate according to the invention. It can be any implant material such as plastics, metals, metal alloys and ceramics. For example, the implant material can be an artificial heart valve of pyrolyzed carbon or a stent such as is described in DE-A-197 53 123.

In one embodiment of the artificial implant according to the invention there is a layer containing an adhesion promoter provided between the surface of the substrate and the biocompatible coating.

The adhesion promoter, or spacer, is, for example, an organosilicon compound, preferably an amino-terminated silane or a compound based on an aminosilane, or an alkylphosphonic acid. Aminopropyltrimethoxysilane is especially preferred.

The adhesion promoter particularly improves the adhesion of the coating to the surface of the implant material through coupling of the adhesion promoter to the surface of the implant material, through, for instance, ionic and/or covalent bonds, and through further coupling of the adhesion promoter to reactive components, particularly to the antithrombogenic polymer of the coating, through, for instance, ionic and/or covalent bonds.

In addition, a process for producing the artificial implants according to the invention is provided, wherein the biocompatible coating is applied to the substrate by reacting the substrate with

(a) a mixture of the antithrombogenic polymer or a precursor of it and the active agent or

(b) the antithrombogenic polymer or a precursor of it to produce a primary polymer coating, and subsequent application/penetration of the active agent into the primary polymer coating.

Especially preferred is a wet chemical process, particularly for process variant (a), because the active agent is often sensitive to drastic reaction conditions. In this case, the substrate is immersed in a solution containing the antithrombogenic polymer and active agent, and optionally the solvent is then removed either by heating or by applying a vacuum. This process is repeated until the coating has the desired thickness.

Suitable solvents for this process are selected from polar aprotic solvents such as esters (such as ethyl acetate, propyl acetate, butyl acetate, ethyl propionate, ethyl butyrate etc.), ketones (such as acetone, ethyl methyl ketone etc.), amides (such as dimethylformamide etc.), sulfoxides (such as DMSO etc.) and sulfones (such as sulfolane etc.). Ethyl acetate is especially preferred. The concentration of the polymer in the solution is 0.001 to 0.5 M, preferably 0.01 to 0.1 M. The concentration of the active agent depends on the desired ratio of polymer to active agent. The immersion time is preferably in the range of 10 seconds to 100 hours. The drying steps are done in vacuum, in air, or in a protective gas in the temperature range, for example, from about −20 ° C. to about 300 ° C., preferably 0 ° C. to 200 ° C., and especially preferably from 20 ° C. to 100 ° C.

The other processes mentioned in DE 196 13 048 can also be used for stable active agents, such as the process of applying polydichlorophosphazene and subsequent reaction with reactive compounds, of melting on, or of sublimation. These processes are usable particularly for the first step of process variant (b), in which the active agent is applied or penetrates in a second step, which second step can then be done preferably by a gentle wet chemical method such as is described above.

In the process using polydichlorophosphazene, a mixture of polydichlorophosphazene and active agent is applied to the surface of the substrate and reacted with at least one reactive compound selected from aliphatic or aromatic alcohols or their salts, alkylsulfones, dialkylamines, and aliphatic or aromatic heterocycles having nitrogen as the heteroatom, corresponding to the definition of R¹ to R⁶, above. The polydichlorophosphazene is preferably applied to the surface of the substrate in an inert gas atmosphere, optionally coupled to the adhesion promoter, and reacted with the reactive compound. Alternatively, polydichlorophosphazene can be applied under reduced pressure or in air, and optionally coupled to the adhesion promoter.

The production of polymers of formula (I), such as poly[bis(trifluoroethoxy)phosphazene], starting with hexachlorocyclotriphosphazene, is known at the state of the art. The polymerization of hexachlorocyclotriphosphazene is described extensively in Korsak et al., Acta Polymerica 30, No. 5, pages 245-248 (1979). Esterification of the polydichlorophosphazene produced by the polymerization is described in Fear, Thower and Veitch, J. Chem. Soc., page 1324 (1958).

In a preferred embodiment of the process according to the invention, an adhesion promoter as defined above is applied to the surface of the substrate before application of the mixture of polymer or polymer precursor and active agent, or before application of polymer or polymer precursor, and coupled to the surface through ionic and/or covalent bonds, for instance. Then the antithrombogenic polymer of polydichlorophosphazene, for example, is applied to the substrate surface coated with the adhesion promoter and is coupled to the adhesion promoter through ionic and/or covalent bonds, for instance.

The adhesion promoter can be applied to the substrate by wet chemistry or in solution or from the melt or by sublimation or spraying. The wet chemical coupling of an adhesion promoter based on amino acids on hydroxylated surfaces, is described in the diploma thesis of Marco Mantar, page 23, University of Heidelberg (1991).

The substrate surface can be cleaned oxidatively, with Caro's acid, for instance, before application of polydichlorophosphazene, the adhesion promoter, or the antithrombogenic polymer. Oxidative cleaning of surfaces with simultaneous hydroxylation, such as can be used, for instance, for implants of plastics, metals or ceramics, is described in Ulman Abraham, Analysis of Surface Properties, “An Introduction to Ultrathin Organic Films”, 108, 1991.

In summary, it has been established that the artificial implants according to the invention surprisingly retain the outstanding mechanical properties of the implant material as the substrate. Due to the coating applied according to the invention, for instance, by direct deposition from the solution, they exhibit not only antithrombogenic but also anti-restenosis properties, drastically improving the biocompatibility and usability of such artificial implants. These surprising results can be demonstrated easily by X-ray photoelectron (XPS) spectra.

The present invention is further illustrated in the following examples.

### EXAMPLES

### Example 1

A: The polydichlorophosphazene on which the poly[bis(trifluoroethoxy)phosphazene] is based, is produced by polymerization of hexachlorocyclotriphosphazene at 250±1° C. in an ampule with a diameter of 5.0 mm and under a pressure of 1.3 Pa (10⁻² mm Hg) prevailing in the ampule. This is done by first preparing a 0.1 M solution of polydichlorophosphazene (0.174 g in 5 ml solvent) in an inert gas atmosphere. Absolute toluene is used as the solvent. Then the esterification is done in this solution with sodium 2,2,2-trifluoroethanolate in absolute tetrahydrofuran as the solvent (8 ml absolute tetrahydrofuran, 0.23 g sodium, 1.46 ml 2,2,2-trifluoroethanol).

B: For oxidative cleaning and simultaneous hydroxylation of the artificial implant surfaces, the substrate is placed in a mixture of 1:3 30% H₂_{O}₂ and concentrated sulfuric acid (Caro's acid) for 2 hours at a reaction temperature of 80° C. After that treatment, the substrate is washed with 0.5 liters deionized water [with a resistivity] of 18 MΩ·cm and about pH 5, and then dried in a stream of nitrogen.

C: To coat the surface of the implant with an adhesion promoter, the artificial implant, oxidatively cleaned with Caro's acid according to Example 1B, is immersed for 30 minutes at room temperature in a 2% solution of aminopropyltrimethoxysilane in absolute ethanol. Then the substrate is washed with 4-5 ml absolute ethanol and left in the drying cabinet for 1 hour at 105° C.

### Example 2

A: An artificial implant pretreated according to Example 1B and 1C was placed for 24 hours at room temperature in a 0.1 M solution of poly[bis(trifluoroethoxy)phosphazene] in ethyl acetate (0.121 g in 5 ml ethyl acetate) which contained 0.0121 g probucol. Then the artificial implant produced in that manner was washed with 4-5 ml ethyl acetate and dried in a stream of nitrogen.

B: An artificial implant pretreated according to Example 1B and 1C was placed for 24 hours at room temperature in a 0.1 M solution of poly[bis(trifluoroethoxy)phosphazene] in ethyl acetate (0.121 g in 5 ml ethyl acetate) which contained 0.0242 g trapidil. Then the artificial implant produced in that manner was washed with 4-5 ml ethyl acetate and dried in a stream of nitrogen.

The surfaces of the artificial implants produced in Examples 2A and 2B were examined by photoelectron spectrometry to determine their elemental composition, their stoichiometry and the coating thickness. The results showed that the poly[bis(trifluoroethoxy)phosphazene] had been successfully immobilized with aminopropyltrimethoxysilane as the adhesion promoter, and that coating thicknesses greater than 2.4 nm were attained. Further, it could also be shown by analysis (NMR) that trapidil or probucol had been embedded in the coating in corresponding proportion.

### Example 3

An artificial implant cleaned according to Example 1B was placed for 24 hours at 70° C. in a 0.1 M solution of poly[bis(trifluoroethoxy)phosphazene] in ethyl acetate (0.121 g in 5 ml ethyl acetate) which contained 0.0121 g probucol. Then the artificial implant treated in that manner was washed with 4-5 ml ethyl acetate and dried in a stream of nitrogen.

The artificial implant prepared in this manner was examined by photoelectron spectrometry to determine its elemental composition, its stoichiometry, and the coating thickness. The results showed that the poly[bis(trifluoroethoxy)phosphazene] had been coupled to the implant surface and coating thicknesses greater than 2.1 nm were attained. Further, it could also be shown that the probucol was embedded in the coating in corresponding proportion.

### Example 4

A: An artificial implant pretreated according to Example 1B and 1C was placed for 24 hours at room temperature in a 0.1 M solution of poly[bis(trifluoroethoxy)phosphazene] in ethyl acetate (0.121 g in 5 ml ethyl acetate). Then the artificial implant prepared in this manner was washed with 4-5 ml ethyl acetate and dried in a stream of nitrogen.

B: The substrate obtained according to Example 4A was immersed for 24 hours at room temperature in a solution of cerivastatin in ethyl acetate (0.0121 g cerivastatin in 5 ml ethyl acetate). After drying in a stream of nitrogen, it was shown analytically that the layer of poly[bis(trifluoroethoxy)phosphazene] contained cerivastatin.

1. Artificial implant comprising an implant material as the substrate and a biocompatible coating applied at least partly to the surface of the substrate, which coating comprises an antithrombogenic polymer having the following general formula (I) 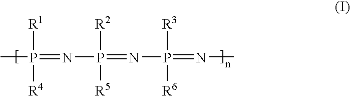
wherein R¹ to R⁶ are the same or different and represent an alkoxy, alkylsulfonyl, dialkylamino or aryloxy group, or a heterocycloalkyl or heteroaryl group having nitrogen as the heteroatom, and at least one other pharmacologically active agent.

2. Artificial implant according to claim 1, wherein at least one of the groups R¹ to R⁶ is an alkoxy group substituted with at least one fluorine atom.

3. Artificial implant according to claim 2, wherein the antithrombogenic polymer is poly[bis(trifluoroethoxy)phosphazene].

4. Artificial implant according to one of the claims 1 to 3, wherein the other pharmacologically active agent is an antimitogenic active agent, especially a cytostatic, a PDGF antagonist, especially trapidil, or a Raf-1 kinase inhibitor.

5. Artificial implant according to one of the claims 1 to 3, wherein the other pharmacologically active agent is a radical trap (especially probucol), an antisense active agent (especially plasmid DNA), a statin (especially cerivastatin) or a GP-IIb/IIIa receptor antagonist (especially abeiximab).

6. Artificial implant according to one of the claims 1 to 5, wherein the ratio of antithrombogenic polymer to active agent is from 1:0.0001 to 1:1, preferably from 1:0.05 to 1:0.5.

7. Artificial implant according to one of the claims 1 to 6, wherein a layer containing an adhesion promoter is provided between the surface of the substrate and the biocompatible coating.

8. Artificial implant according to claim 7, wherein the adhesion promoter is an organosilicon compound, especially aminopropyltrimethoxysilane.

9. Process for producing an artificial implant according to one of the claims 1 to 8, wherein the biocompatible coating is applied to the substrate by reacting the substrate with
(a) a mixture of the antithrombogenic polymer or a precursor of it and the active agent or
(b) the antithrombogenic polymer or a precursor of it, to produce a primary polymer coating, and subsequent application/penetration of the active agent into the primary polymer coating.

10. Process according to claim 9, wherein the coating is applied by wet chemistry, particularly by reacting the substrate with a mixture of antithrombogenic polymer and active agent.

11. Process according to claim 10, wherein the solvent for the wet chemical application is selected from dipolar aprotic solvents and is preferably ethyl acetate.

12. Process according to claims 9 to 11, wherein an adhesion promoter is applied to the surface of the substrate before the biocompatible coating is applied.
